(19) United States
(12) Patent Application Publication
(10) Pub. No.: US 2012/0143060 A1
(43) Pub. Date: Jun. 7, 2012

(54) ULTRASOUND TRANSDUCER ASSEMBLY WITH IMPROVED THERMAL BEHAVIOR

(75) Inventors: Johannes Wilhelmus Weekamp, Beek en Donk (NL); Gideon Frederik Maria Wiegerinck, Eindhoven (NL); Remco Yuri Van de Moesoijk, Geldrop (NL); Richard Davidsen, Andover, MA (US)

(73) Assignee: KONINKLIJKE PHILIPS ELECTRONICS N.V., Eindhoven (NL)

(21) Appl. No.: 12/810,346

(22) PCT Filed: Dec. 22, 2008
(86) PCT No.: PCT/IB2008/055498

§ 371 Date: Jun. 24, 2010

Related U.S. Application Data
(60) Provisional application No. 61/017,044, filed on Dec. 27, 2007.

(51) Int. Cl. A61B 8/00; H05K 13/00

(52) U.S. Cl. 600/459; 216/13; 216/20

(57) ABSTRACT

A transducer assembly (10) is provided that includes a housing (12), a lens (14), an array of transducer elements (18), an interposer assembly (22), a transducer array 5 control assembly (30), and a heat sink assembly (32). The interposer assembly (22) includes a plurality of signals tracks (56) that provide electrical connections between the array of transducer elements (18) and the transducer array control assembly (30). The interposer assembly (22) further includes heat transporter bars (50) for transporting heat within the interposer (22) to the heat sink assembly (32). A flexible interconnection 10 assembly (28) is disposed between the interposer assembly (22) and the transducer array control assembly (30) providing re-workable electrical connections between the signal tracks (56) of the interposer assembly (22) and the transducer array control assembly (30).

### TECHNICAL FIELD

The present disclosure relates to systems for ultrasonic diagnostic imaging. More particularly, the present disclosure is directed to ultrasonic apparatus/systems and related methods that include and/or facilitate use of both large and small arrays of transducer elements in ultrasound transducer probes.

### BACKGROUND

Ultrasonic diagnostic imaging systems allow medical professionals to examine internal structures of patients without invasive exploratory surgery. Ultrasonic diagnostic imaging systems typically include a transducer probe connected to a host system that provides control signals to the transducer probe, processes data acquired by the transducer probe, and displays a corresponding image.

Current transducer probes generally consist of a row of transducer elements, each of which is connected to a terminal of a transducer control assembly or application specific integrated circuit (ASIC) that processes signals transmitted to and received from the acoustic elements. Typically, such connections are made by soldering wires disposed at one end of a flex-cable to the individual transducer elements. The other end of the cable is generally connected to a console with all the signal processing electronics. Typically, 96 to 256 transducer elements are arranged at pitches that vary from 150 to 500 microns.

Next generation transducers are expected to employ arrays of several thousands of transducer elements arranged in a matrix configuration, such matrix configuration consisting of multiple rows and columns of transducer acoustic elements. Each transducer element requires an electrical interconnection to a terminal of the ASIC (or other control circuit). The large amount of transducer elements would necessarily require a very large cable with thousands of wire strands, raising significant issues of practicality.

An interposer consisting of a block of backing material with parallel signal tracks disposed therein could be used to interconnect terminals of the ASIC and signal lines connected to individual transducer elements. For example, one such interposer is disclosed in commonly assigned U.S. Provisional Patent Application No. 60/820,184, filed Jul. 24, 2006, the disclosure of which is herein incorporated by reference. The previously disclosed interposer matches differences in pitch between terminals of the ASIC and signal contacts leading to the individual transducer elements. Using the previously disclosed interposer, a standardized ASIC could be used for different transducer array geometries.

However, building a transducer probe having a large number of transducer elements presents many design challenges. Current ASIC designs only accommodate connection with a few hundred transducer elements. Thus, a transducer probe containing thousands of transducer elements exceeds the connection capacity of conventional ASIC designs, thereby requiring several ASICs. Further, since transducer elements are generally fabricated, at least in part, from expensive piezoelectric materials, it is important to have a reliable interconnection process between pre-tested ASICs and any interposer on which the transducer elements are mounted. If a fixed interconnection structure, such as conductive glue, is used, it is not possible to efficiently rework the device in the event of a component failure. A re-workable interconnection technique/assembly that facilitated ASIC to transducer elements connection would provide an economic solution by providing for the disassembly and re-assembly of the transducer probe, if necessary.

There are generally two primary heat sources within a housing of the transducer probe that must be addressed. First, part of the acoustic power generated by the transducer elements is lost as heat generation in the acoustic stack. The majority of this heat is created within the lens of the transducer probe and is typically on the order of one Watt. Second, in operation, each ASIC typically dissipates about 1 Watts of heat. Additional heat sources may be present, e.g., electronics associated with wireless transmission. Of note, in transducer designs that include a plurality of ASICs, e.g., “N” ASIC elements, the total heat generated in a transducer can be “N” times the power generated in an individual ASCI associated with such plurality of ASICs. Thus, effective transducer designs must take account of potential heat effects.

There are restrictions on the maximum transducer lens temperature that may be permitted/accommodated because the transducer lens contacts a human body during an examination. Thermal design considerations are of increasing importance with respect to next generation ultrasound transducer probes, e.g., to prevent the lens temperature from becoming excessive during operation of the transducer probe. Issues may arise with current techniques for passive heat removal, which generally rely upon heat convection to the environment in combination with heat conduction through a cable between the transducer probe and the host system, particularly as next generation transducer probes are developed and commercialized. The effectiveness of passive heat removal generally depends on factors specific to the transducer design, e.g., the transducer lay-out (which may directly impact heat dissipation) and available heat rejection surface areas. For effective passive heat removal, heat is ideally well distributed over the transducer housing.

The patent literature includes teachings of background relevance. For example, U.S. Pat. No. 6,589,180 to Erikson et al. discloses a high density ultrasound transducer array using multi-layer structures composed of active integrated circuit devices on various substrates and passive devices. Electrically conducting interconnections between substrates are implemented with micro-vias configured with conductors extending through the substrates. The various layers may be assembled with solders that permit testing of selected layers and circuits prior to completion. Similarly, U.S. Pat. No. 5,629,578 to Winzer et al. discloses a transducer array that is packaged in a high density interconnected multi-chip module which has the integrated circuit chips disposed in a substrate, interconnection layers disposed thereon and multilayer composite actuators disposed on the surface of the interconnection structure.

U.S. Pat. No. 6,859,984 to Dinet et al. discloses a method for producing a matrix array ultrasonic transducer having an integrated interconnection assembly. A piezoelectric member formed by a plurality of individual elemental transducers arranged in a matrix configuration is provided and an interconnect interface is joined to the rear face of the piezoelectric member. The interconnect device is formed by an insulator member having dimensions in accordance with those of the piezoelectric member. A drilling operation is performed on the insulator member to form a corresponding array of through holes. The insulator member is then metallized and a resin used to provide filling of the through holes. See, also, U.S. Pat. No. 4,864,179 to Lapetina et al., U.S. Patent Publication No. 2005/0075573 to Park et al., and U.S. Patent Publication No. 2006/0043839 to Wildes et al.

The noted patent literature fails to address several shortcomings of the prior art that are addressed in the present disclosure, including, inter alia, the need in ultrasound transducer design/fabrication to establish reliable contact between piezoelectric arrays and ASICs while simultaneously permitting de-mating, e.g., if a replacement ASIC is required. Thus, despite efforts to date, new designs, systems and methods are needed to accommodate next generation ultrasound transducer probes, particularly with respect to the issues and limitations noted above.

### SUMMARY

The present disclosure provides advantageous designs, systems and methods for employing a large array of transducer elements within a transducer assembly. The transducer assembly typically includes a housing, a lens, an array of transducer elements, an interposer assembly, and a transducer array control assembly. Exemplary interposer assemblies according to the present disclosure include a plurality of signal tracks that provide electrical connections between the array of transducer elements and the transducer array control assembly. The interposer assembly further includes heat transporting bars/conduits that transport heat from within the interposer originating from the lens and partly from the heat generated within the one or more ASIC's associated with the disclosed transducer assembly. A flexible and/or de-matable interconnection assembly is advantageously disposed between the interposer assembly and the transducer array control assembly to provide and/or facilitate re-workable electrical connections between the signal tracks of the interposer assembly and the transducer array control assembly.

In some disclosed embodiments, the transducer assembly includes one or more air gaps between the ASIC(s) and the acoustic stack associated with the disclosed transducer assembly. The air gap(s) provide a thermal barrier therebetween. Signal tracks across such air gap(s) are generally provided, e.g., in a thin/ultra-thin Parlyene™ film (polyxylene polymer marketed by Para Tech Coating, Inc., Aliso Viejo, Calif.). In further disclosed embodiments, heat removal strips are disposed at opposing ends of the array of transducer elements to provide temperature control functionality. The heat removal blocks of exemplary embodiments of the present disclosure are effective to prevent the lens temperature from becoming excessive, e.g., surpassing a predetermined level.

Exemplary interposer assemblies of the present disclosure include at least first and second regions. For example, the first region may be fabricated from a first material disposed with respect to (e.g., in juxtaposition with) the transducer control assembly and a second region fabricated from a second material disposed with respect to (e.g., in juxtaposition with) the array of transducer elements. The first material creates a thermal barrier that prevents heat generated by the transducer control assembly from migrating toward the lens. The second material absorbs acoustic energy generated by the array of transducer elements.

In some embodiments, the interposer includes one or more air gap(s) between regions/materials disposed with respect to the transducer control assembly and regions/materials disposed with respect to the transducer control assembly. The disclosed air gap(s) may function to create an additional thermal barrier that prevents heat generated by the transducer control assembly from migrating towards the lens and directs heat originating from the lens towards a separate/distinct heat rejection area.

Of note, in exemplary embodiments of the present disclosure, the transducer assembly includes at least two heat rejection/removal areas: one heat rejection area is effective to reject/remove lens heat, and the second heat rejection area is effective to reject/remove ASIC-generated heat. The disclosed thermal barrier (e.g., one or more air gaps) is generally effective to prevent at least the majority (if not all) of the ASIC-generated heat from flowing to the lens. Another function of the thermal barrier is to direct the heat from the lens to the heat transporter bar so that such heat can flow to its own “heat rejection area” as disclosed herein.

Additional features, functions and benefits of the disclosed designs, assemblies and methods will be apparent from the description which follows, particularly when read in conjunction with the accompanying figures.

### BRIEF DESCRIPTION OF THE DRAWINGS

To assist those of skill in the art in making and using the disclosed transducer assemblies and related methods, reference is made to the accompanying figures, wherein:

FIG. 1A is a schematic depiction of an exemplary ultrasonic transducer assembly made in accordance with the present disclosure;

FIG. 1B is a schematic illustration of an exemplary transducer assembly that provides active cooling functionality according to the present disclosure;

FIG. 1C is a schematic illustration of an exemplary transducer assembly that provides passive cooling functionality according to the present disclosure;

FIG. 1D is a schematic illustration of an exemplary transducer assembly with enlarged schematic cross section of lens region;

FIG. 2 is a schematic depiction of a metallic plate used in the construction of the exemplary ultrasonic transducer assembly shown in FIG. 1;

FIGS. 3A-3F depict a process for fabricating an interposer according to an embodiment of the present disclosure;

FIGS. 4A-4F depict an alternative process for fabricating an interposer according to an embodiment of the present disclosure;

FIGS. 5A-5G depict a process for fabricating an interposer according to another embodiment of the present disclosure;

FIGS. 6A-6G depict a process for fabricating an interposer according to another embodiment of the present disclosure;

FIGS. 7A-7E depict a process for fabricating an interposer according to yet another embodiment of the present disclosure;

FIGS. 8A-8B depict a process for assembling the transducer assembly shown in FIG. 1;

FIGS. 9A-9I depict a process for fabricating a flexible interconnection assembly according to an embodiment of the present disclosure;

FIGS. 10A-10C depict a process for assembling the transducer assembly shown in FIG. 1;

FIGS. 11A-11F depict a process for fabricating a flexible interconnection assembly according to an embodiment of the present disclosure;

FIG. 12 depicts a flowchart for an exemplary fabrication method according to the present disclosure;

FIG. 13 depicts a three-step flowchart showing assembly of a “flex-pad” fabricated according to the method of FIG. 12 in combination with an ASIC and an interposer; and

FIG. 14 depicts exploded views (top and bottom views) of an exemplary transducer subassembly according to the present disclosure.

### DESCRIPTION OF EXEMPLARY EMBODIMENT(S)

In accordance with the exemplary embodiments of the present disclosure, an ultrasound transducer probe is provided for anatomical imaging. The disclosed ultrasound transducer probe may support active cooling, passive cooling or a combination thereof. Thus, the disclosed transducer probe may include a housing, a lens, a high density array of transducer elements, a heat transporting interposer, a heat sink, and a flexible and/or de-matable interconnection assembly. Elements/components are included in the disclosed transducer probe so as to achieve desired heat removal/rejection functionalities.

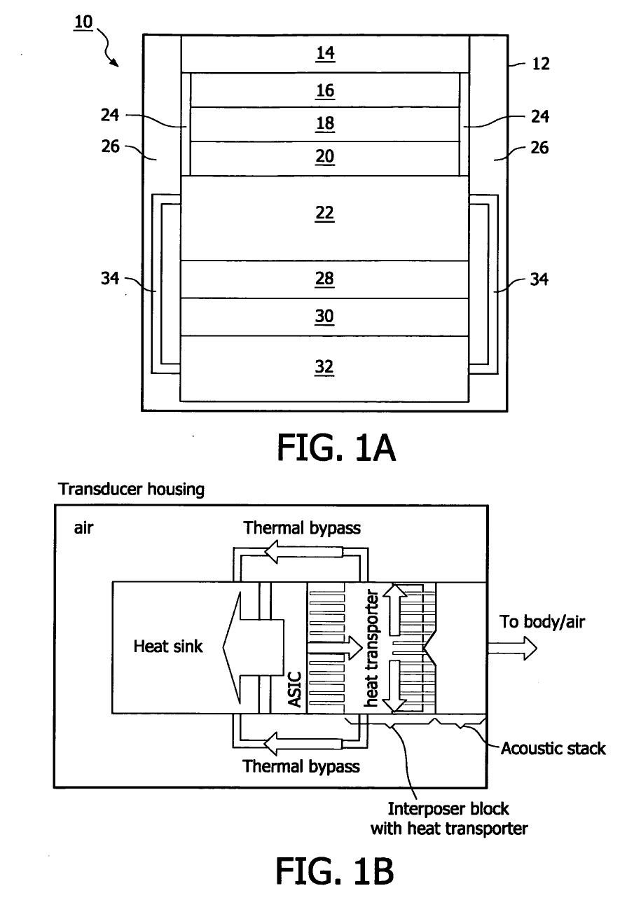

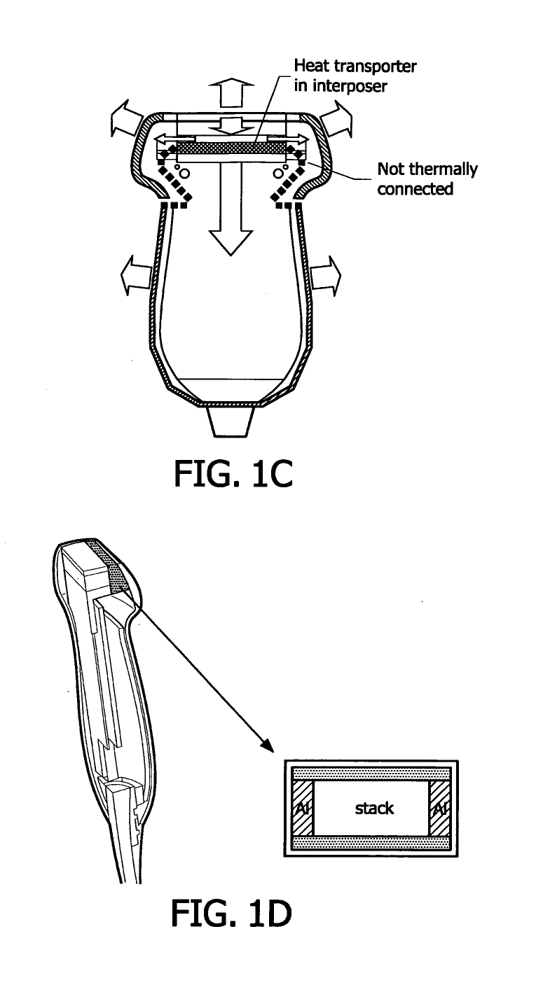

Referring now to FIG. 1A, an exemplary ultrasound transducer probe is generally indicated at 10. The ultrasonic transducer probe 10 includes a housing 12 having a lens 14 disposed with respect to the housing 12. A matching layer 16 is disposed between the lens 14 and an array of transducer elements 18. A dematching layer 20 is disposed between the array of transducer elements 18 and an interposer assembly 22. The interposer assembly 22 contains signal tracks (not shown), each of which is in electrical communication with one of the transducer elements of array 18. The matching layer 16, array of transducer elements 18, and the dematching layer 20 are collectively referred to herein as the acoustic stack.

With further reference to the schematic illustration of FIG. 1A, heat removal blocks 24 are disposed at opposing sides of the interposer assembly 22 between the acoustic stack and the one or more ASICs. The heat removal blocks 24 are preferably formed from aluminum and are about one and one-half millimeters in width, although alternative materials of construction and dimensional parameters may be employed without departing from the spirit or scope of the present disclosure. For example, copper and/or composite materials may be employed in place of aluminum, but processing of such alternative materials may prove difficult and/or infeasible. Air gaps 26 are formed between the heat removal blocks 24 and the housing 12 of the transducer assembly 10.

With reference to FIG. 1B, a schematic depiction of a transducer assembly that provides active cooling functionality is provided. The transducer housing includes a cooled heat sink that is in thermal communication with the ASIC(s) and an interposer block with heat transporter. Thus, heat generated by the one or more ASIC's associated with the transducer assembly and positioned within the housing flows directly to the heat sink. In addition, heat from acoustic losses within the matching layers and lens flow via the heat transporter and thermal bypasses to the heat sink. In this way, active cooling of the transducer assembly may be advantageously achieved.

Turning to FIG. 1C, a further exemplary transducer probe assembly according to the present disclosure is schematically depicted. The transducer probe of FIG. 1C advantageously facilitates “passive cooling” to address heat generated therewithin. Thus, as shown in FIG. 1C, the disclosed transducer probe is designed such that heat from acoustic losses within the matching layers and lens are partly rejected by the lens at the lens surface (upward vertically directed arrow). A further portion of heat associated with acoustic losses flows through acoustic stack to heat transporter functionality within an interposer element. Such acoustic loss-related heat is transported to the sides of the housing and rejected over the nose of the transducer assembly. (downward and outwardly directed arrows in the nose region).

According to the exemplary transducer assembly of FIG. 1C, heat generated in the one or more ASICs is also passively dissipated. Only a small amount of ASIC-generated heat flows to the lens due to the large thermal flow resistance associated with the interposer positioned therebetween. Accordingly, a majority of the ASIC-generated heat is rejected over the surface of the handle of the transducer probe assembly, as schematically depicted by the downward and outwardly directed arrows in the handle region of the transducer probe. As schematically depicted by the broken/dotted lines in FIG. 1C, the top part (i.e., nose region) of the transducer assembly is advantageously thermally disconnected and/or isolated from the handle region thereof. In this way, ASIC-generated heat is effectively isolated from the lens region of the transducer probe and substantially limited in its flow path to the handle surface.

Returning to FIG. 1A, a flexible interconnection assembly 28 may be advantageously disposed between the interposer assembly 22 and a transducer control assembly 30. The flexible interconnection assembly 28 forms electrical connections with contact portions (not shown) formed on the signal tracks of the interposer assembly 22 and contact portions (not shown) formed on a surface of the transducer control assembly 30. For implementations that include active cooling functionality, as described herein, a heat sink assembly 32 may be disposed on the opposite side of the transducer control assembly 30. In such implementations, heat bypasses/conductors 34 conduct heat from the interposer assembly 22 to the heat sink assembly 32.

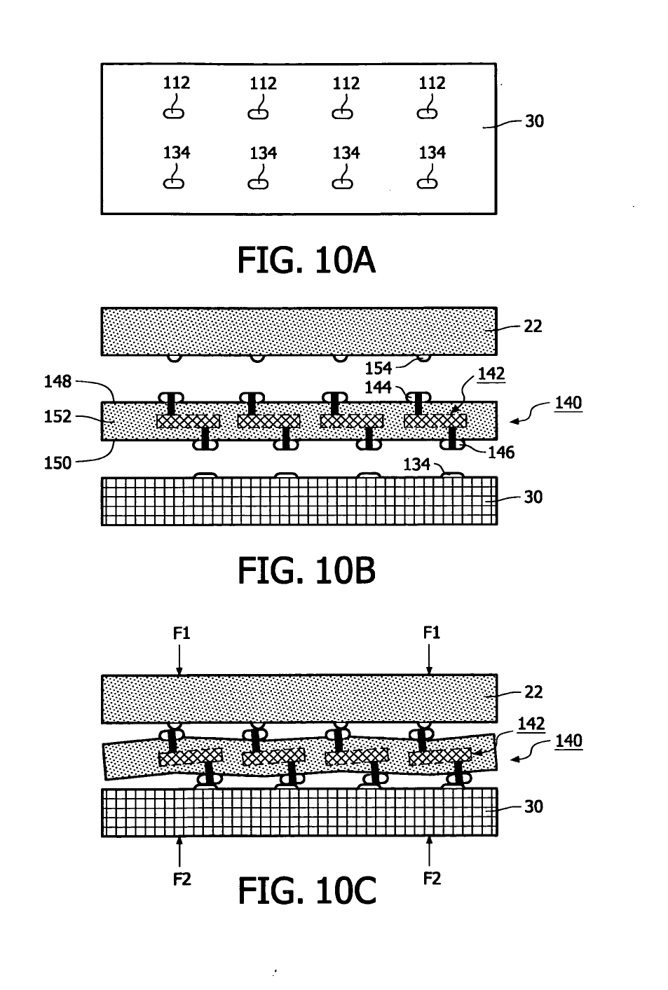

With reference to FIG. 10, an enlarged schematic cross-sectional view of the lens region of an exemplary transducer assembly is provided. As shown therein, an aluminum block may be employed within the acoustic stack (instead of lens material at short sides of transducer) to enhance the connection therewithin.

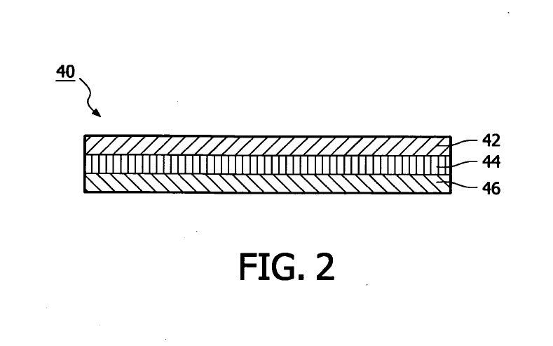

Construction of an exemplary interposer assembly 22 is described with reference to FIGS. 2-4. With particular reference to FIG. 2, fabrication of exemplary interposer assembly 22 begins with a metallic stack 40. The metallic stack includes a first copper layer 42 (e.g., about twenty-five microns in thickness), a nickel layer 44 (e.g., about two microns in thickness), and a second copper layer 46 (e.g., about sixty-five microns in thickness).

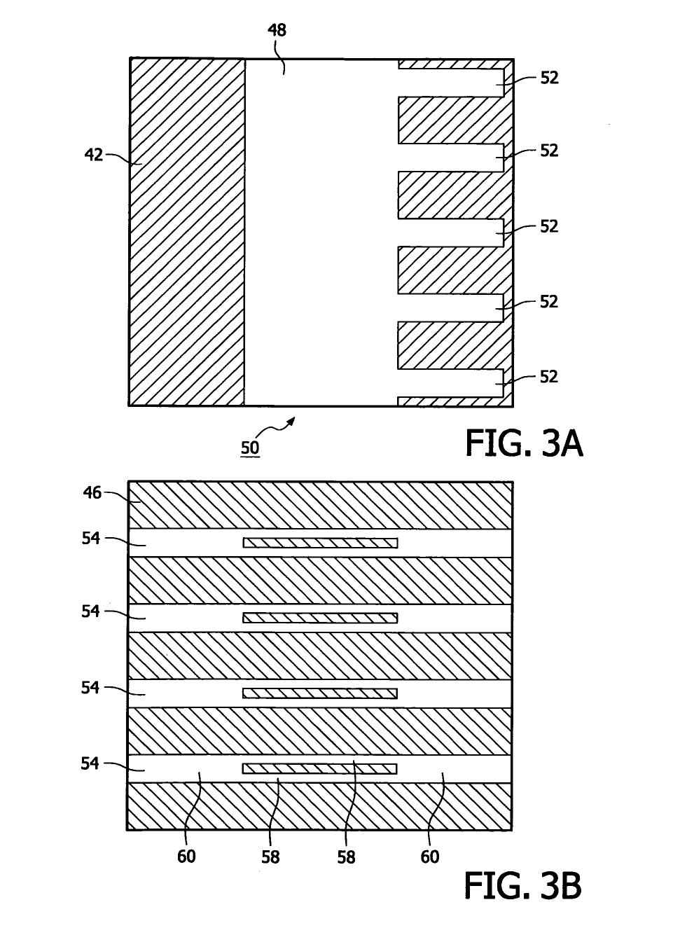

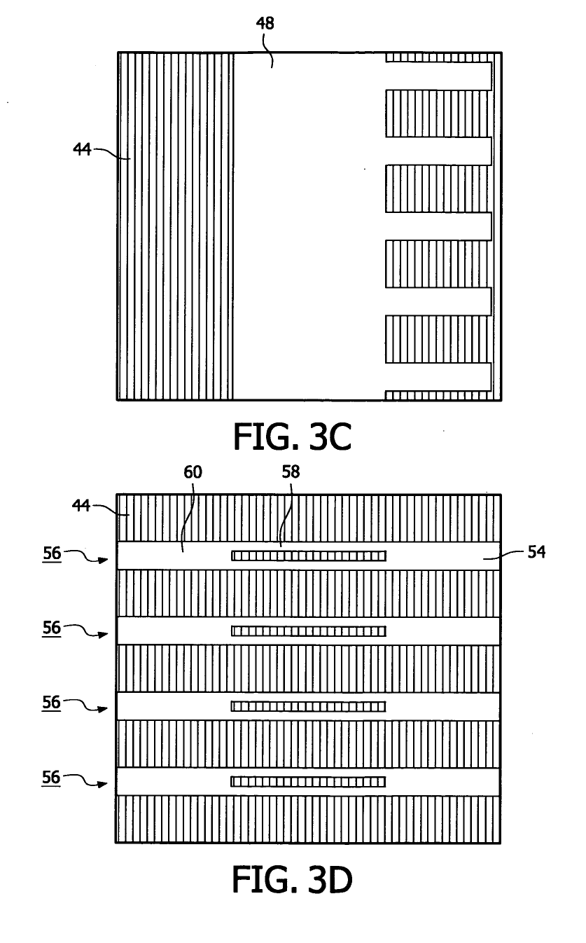

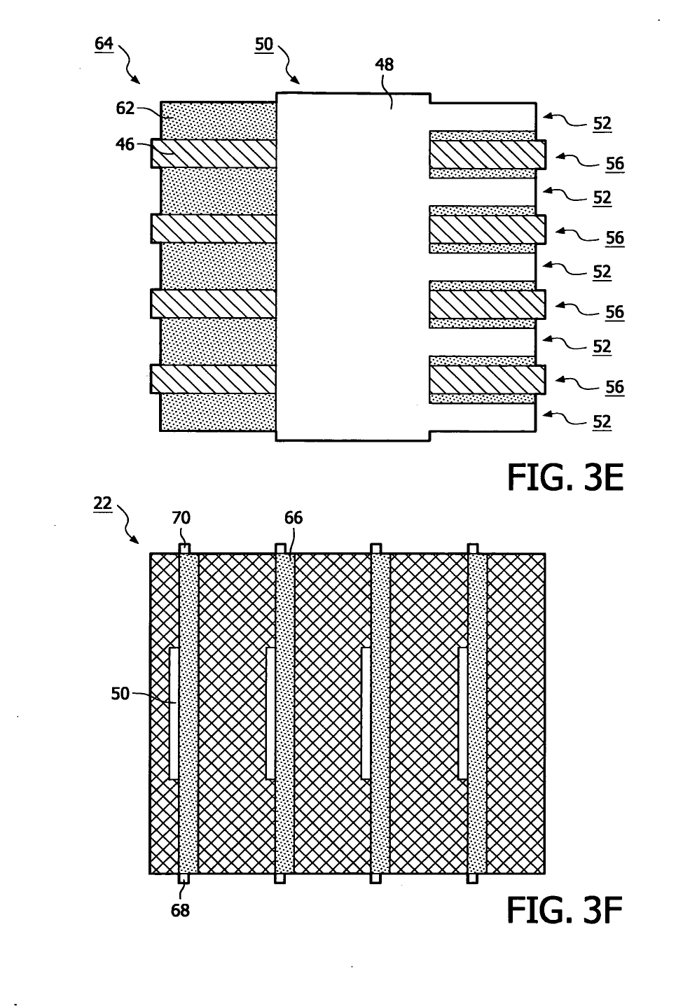

A first fabrication process for forming the interposer assembly 22 is described with reference to FIGS. 2 and 3A-3F. Referring now to FIGS. 2 and 3A, a first nickel/gold layer 48 (e.g., about two microns and one micron in thickness, respectively) is electroplated onto a relatively thick member 46 that is typically fabricated from copper. The first nickel/gold layer 48 forms/defines a heat transporter bar 50 and, in exemplary embodiments, a plurality of heat transporter fingers 52. The heat transporter fingers 52 (when present) generally function to improve the thermal path and/or flow for heat originating from the lens, but effective heat removal/rejection may be achieved according to the present disclosure without inclusion of such heat transporter fingers 52.

Referring now to FIG. 3B, a second nickel/gold layer 54 (e.g., about two microns and one micron in thickness, respectively) is electroplated onto the exposed surface of a relatively thin copper layer 42 associated therewith. The second nickel/gold layer 54 forms/defines a plurality of signal tracks 56, each of which includes narrow first portions 58 and wider second portions 60.

A first etching process is performed to remove exposed portions of the first copper layer 42, thereby exposing one side of the nickel layer 44, as shown in FIG. 3B After placement of backing material and epoxy strips, as described herein, a second etching process is performed to remove exposed portions of the second copper layer 46, thereby exposing the opposite side of the nickel layer 44, as shown in FIG. 3C. It is noted that the first portions 58 of the signal tracks 56 may be dimensioned (e.g., approximately 25 microns wide) such that all of the first copper layer 42 below the first portions 58 is fully removed. As a result, the narrow portions 58 of the signal tracks 56 are suspended just above the nickel layer 44. In contrast, the wider portions 60 of the signal tracks 56 retain a portion of the first copper layer 42 between the second nickel/gold layer 54 and the nickel layer 48.

Backing material 62 and epoxy strips (both e.g., about 250 microns in thickness) are typically adhered to the signal tracks 56 (shown in FIG. 3D) with a glue epoxy. The epoxy strips are generally characterized by a low coefficient of thermal expansion and are well fitted to connect to the control ASICs. The backing material 62 helps to absorb sound waves generated by the array of transducer elements 18 (shown in FIG. 1A). Suitable backing materials include highly filled epoxies, wherein filler materials, such as heavy metal oxides and hollow glass spheres, determine acoustic properties of the backing material. Thus, a second etching process may be performed to remove the exposed copper layer 46 and a third etching process may be performed to remove the exposed nickel layer 44.

Underfill material 66 is used to adhere a plurality of interposer layers 64 to form exemplary interposer assembly 22, as shown in FIG. 3F. Underfill material 66 is advantageously formed from a low viscosity epoxy, such as Namix Chipcoat 8462-21, for example. Contact portions 68, 70 are then added to the interposer assembly 22. For example, on the side to be connected with the transducer control assembly 30, a metallization step over the bottom surface of the interposer may be performed using thin film metallization with gold. On the side to be connected to the acoustic stack, a nickel layer (e.g., about 10 microns in thickness) may be electroplated to end portions of the signal tracks 56 (not shown). The nickel layer may then be electroplated with a gold layer (e.g., about one micron in thickness) to form contact portions 70. The contact pads 68 on the bottom side of the interposer may be formed by dicing or the like.

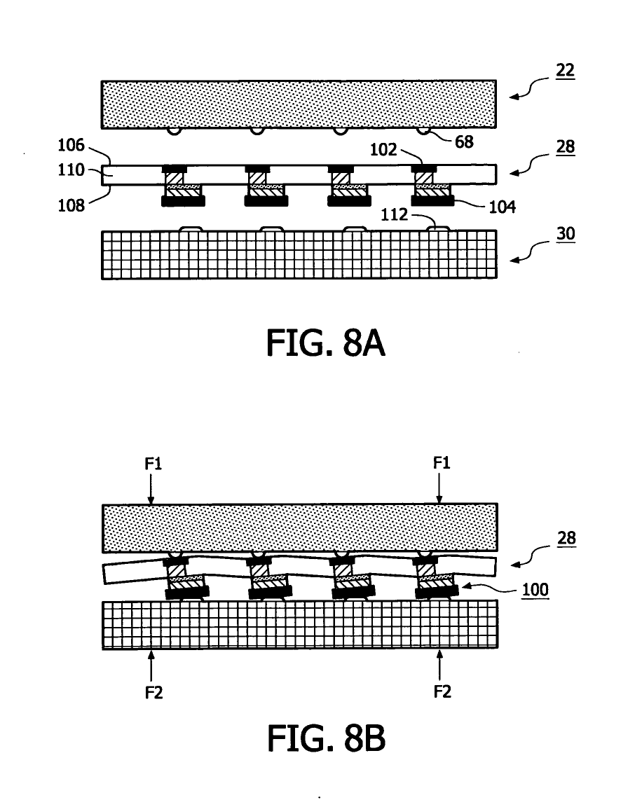

The thickness of the underfill material 66 is selected to space the contact portions 68 in correspondence with contact portions 102 of exemplary flexible interconnection assembly 28 (shown in FIG. 8A), as will be described below in detail. In addition, heat bypasses 34 (shown FIG. 1) generally communicate with portions of the heat transporter bars 50 that extend past the backing layer 60. For example, the heat bypasses 34 may be soldered or otherwise connected, e.g., with an adhesive, a thermal interface material, or another material offering similar functionality, to protruding ends of the heat transporter bars 50. Alternatively, the heat bypasses 34 may be adhered to protruding ends of the heat transporter bars 50 with a thermally conductive glue, a thermal interface material or another material/approach offering similar functionality.

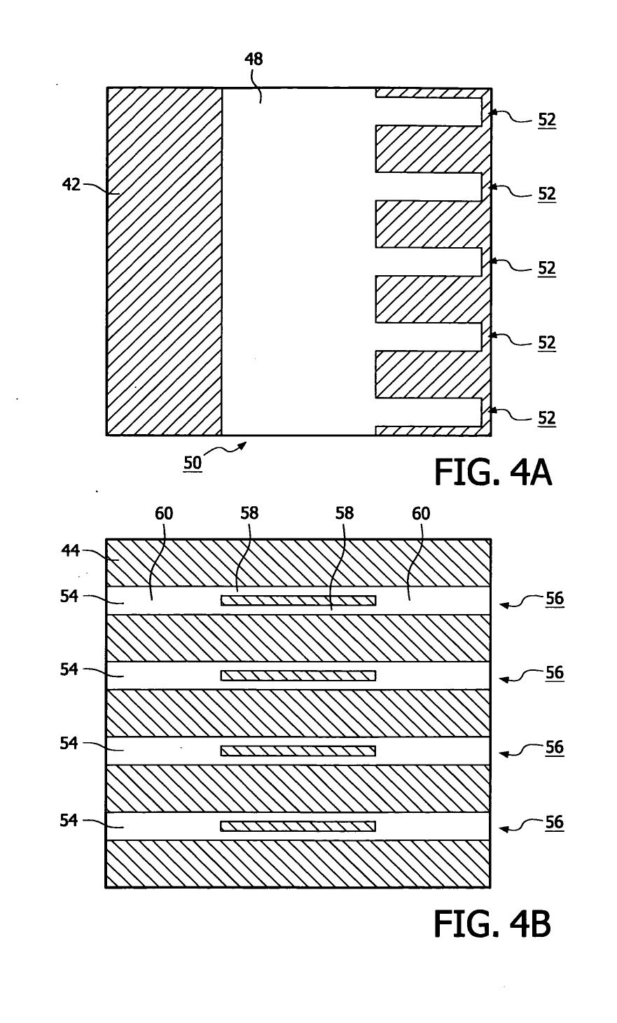

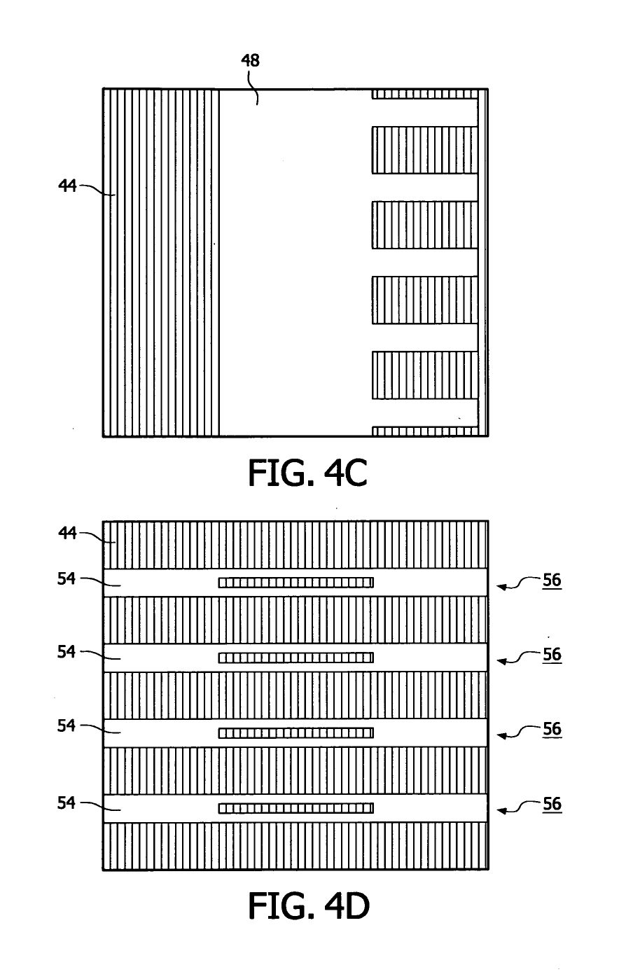

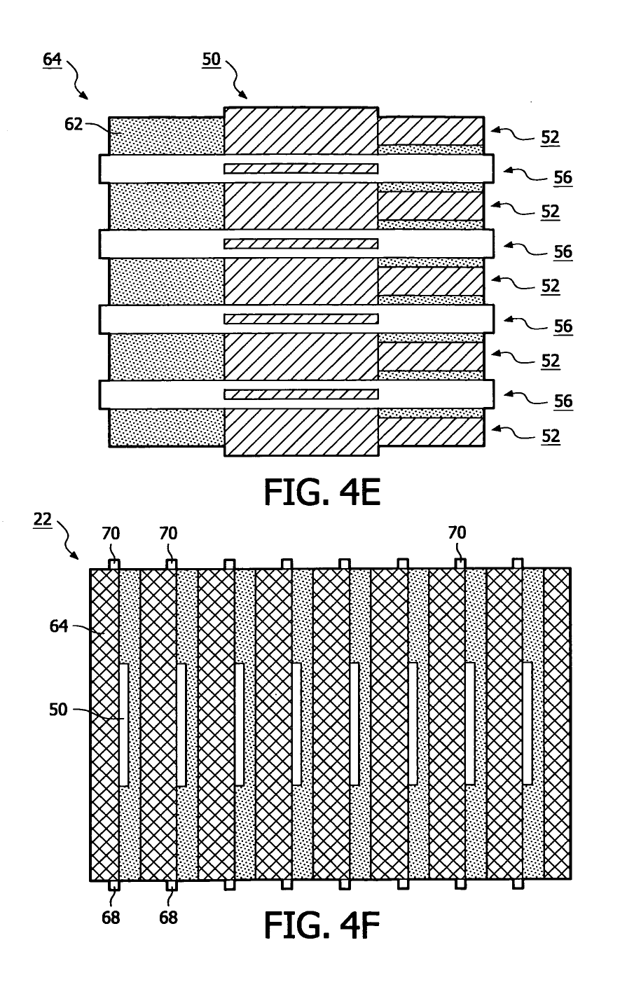

A second fabrication process for forming an exemplary interposer assembly 22 according to the present disclosure is described with reference to FIGS. 2 and 4A-4F. Referring now to FIGS. 2 and 4A, a first nickel/gold layer 48 (e.g., about two microns and one micron in thickness, respectively) is electroplated onto the exposed surface of the first copper layer 42. The first nickel/gold layer 48 forms/defines a heat transporter bar 50 and a plurality of heat transporter fingers 52. Referring now to FIG. 4B, a second nickel/gold layer 54 (e.g., about two microns and one micron in thickness, respectively) is electroplated onto the exposed surface of the second copper layer 46. The second nickel/gold layer 54 forms/defines a plurality of signal tracks 56, each of which includes narrow first potions 58 and wider second portions 60.

A first etching process is performed to remove exposed portions of the first copper layer 42, thereby exposing one side of the nickel layer 44, as shown in FIG. 4C. Backing material 62 and epoxy strips (e.g., both about 250 microns in thickness) may be adhered to the signal tracks, e.g., with a glue epoxy. After placement of the backing material and epoxy strips, a second etching process is performed to remove exposed portions of the second copper layer 46, thereby exposing the opposite side of the nickel layer 44, as shown in FIG. 4D. As described above, the backing material generally helps to absorb sound waves generated by the array of transducer elements 18. Suitable backing materials include highly filled epoxies, wherein filler materials, such as heavy metal oxides and hollow glass spheres, determine acoustic properties of the backing material. A third etching process may be performed to remove the nickel layer 44.

As previously described with reference to an alternative implementation of the present disclosure, underfill material 66 material may be adhered to a plurality of interposer layers 64 to form exemplary interposer assembly 22, as shown in FIG. 4F. Contact portions 68, 70 are then added to the interposer assembly 22. Heat bypasses may be attached to or otherwise placed in thermal communication with portions of the heat transporter bars 50 that extend past the backing layer 62 and to the heat sink assembly 32.

A second fabrication method is disclosed herein which is preferred for certain applications because the second copper layer 46 may be fabricated with a smaller thickness, e.g., 25 microns, as compared to the exemplary 65 microns described with reference to the first fabrication method described herein. The resultant thinner gaps between the signal tracks 56 and the heat transporter bar 50 may be advantageous in certain ultrasound applications of the present disclosure. For example, more backing material 62 may be employed and better acoustical performance achieved according to the second disclosed fabrication method.

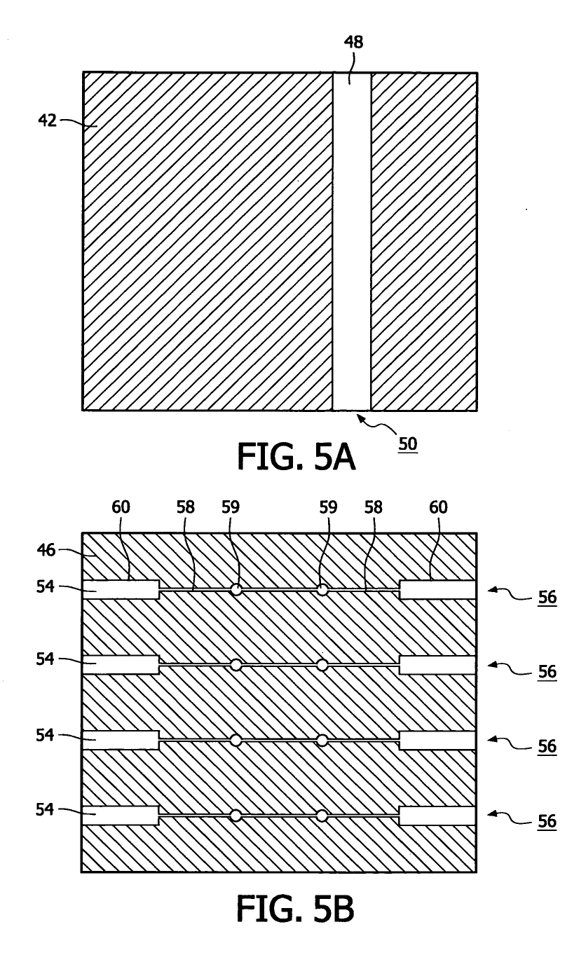

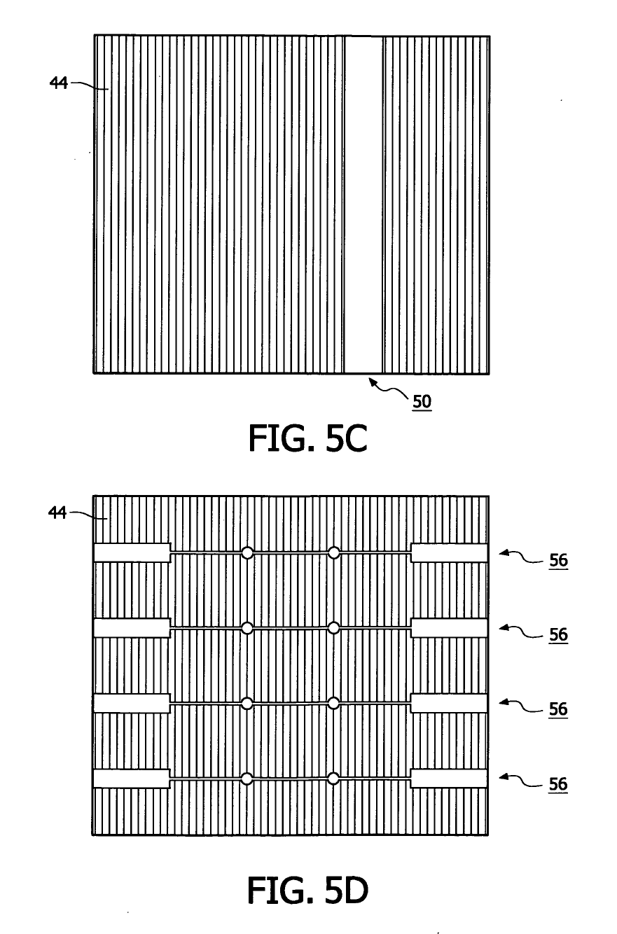

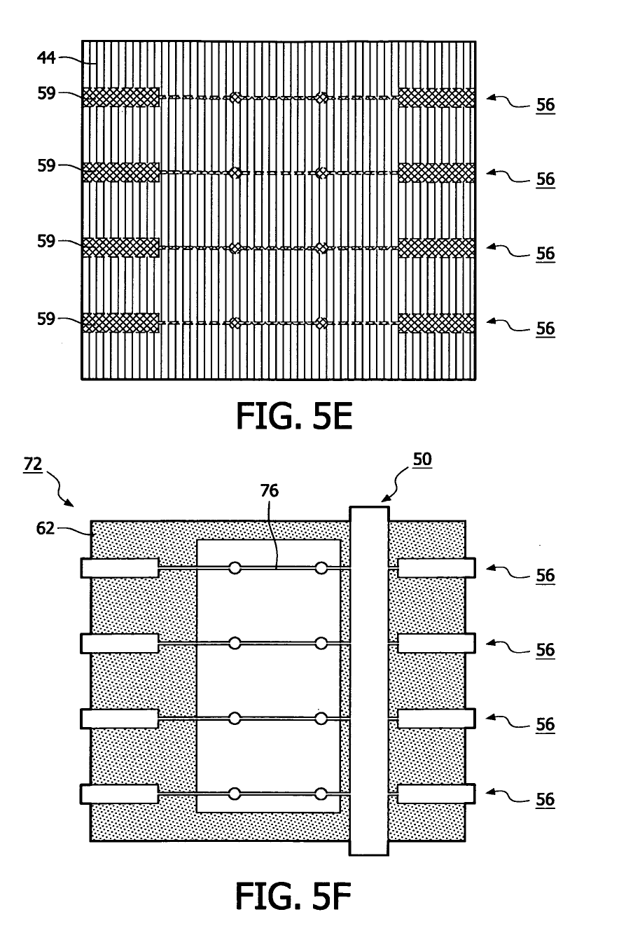

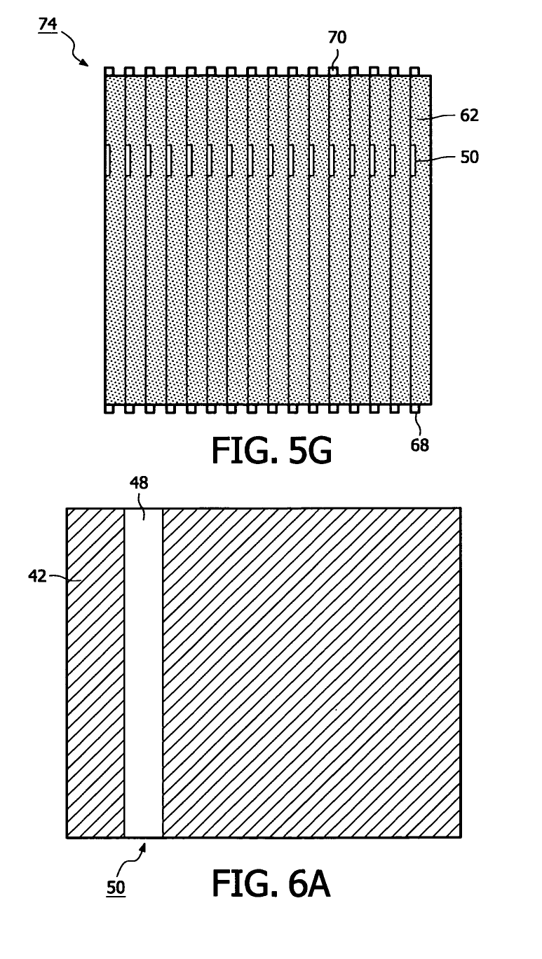

Thus, an alternative exemplary interposer fabricated according to the second fabrication method of the present disclosure is described with reference to FIGS. 2 and 5A-5F. Referring now to FIGS. 2 and 5A, a first nickel/gold layer 48 (e.g., about 2 microns and one micron in thickness, respectively) is electroplated onto the exposed surface of the first copper layer 46. The first nickel/gold layer 48 forms/defines a heat transporter bar 50. Referring now to FIG. 5B, a second nickel/gold layer 54 (e.g., about 2 microns and one micron in thickness, respectively) is electroplated onto the exposed surface of the second copper layer 42. The second nickel/gold layer 54 forms a plurality of signal tracks 56, each of which includes narrow first portions 58 and wider second portions 59 and third portions 60.

A first etching process is performed to remove exposed portions of the first copper layer 42, thereby exposing one side of the nickel layer 44, as shown in FIG. 5C. A thin/ultra-thin Parylene™ layer 59 having a thickness of about five microns may be advantageously applied to the signal tracks 56, as shown in FIG. 5E. An epoxy frame may be adhered/glued on top of signal tracks 56

A second etching process is performed to remove exposed portions of the second copper layer 46, thereby exposing the opposite side of the nickel layer 44, as shown in FIG. 5C. It is noted that the first portions 58 of the signal tracks 56 in the exemplary embodiment described herein are approximately 25 microns wide; thus, all of the second copper layer 46 below the first portions 58 is fully removed. As a result, the first portions 58 of the signal tracks 56 are suspended just above the nickel layer 44. In contrast, the wider second and third portions 59, 60 of the signal tracks 56 retain a portion of the second copper layer 46 (not shown) between the second nickel/gold layer 54 and the nickel layer 44A layer 72 of the interposer 74 is shown in FIG. 5F. In exemplary embodiments of the present disclosure, an air gap 76 may be defined between strips of backing material 62 adjacent one end of the signal tracks 56 and strips of backing material 62 adjacent the other end of the signal tracks 56. The air gap(s) 76 advantageously define a further thermal barrier for purposes of the disclosed interposer assembly.

A low coefficient of thermal expansion epoxy is generally employed to adhere a plurality of interposer layers 72 to form exemplary interposer 74, as shown in FIG. 5G. Contact portions 68, 70 are then added to the interposer assembly 22. The thickness of the backing material 62 is selected to space the contact portions 68 in correspondence with contact portions 102 of the flexible interconnection assembly 28 (shown in FIG. 8A), as will be described below in detail. In addition, heat bypasses 34 (shown in FIG. 1) are attached to or otherwise in thermal communication with portions of the heat transporter bars 50 that extend past the backing layer 62.

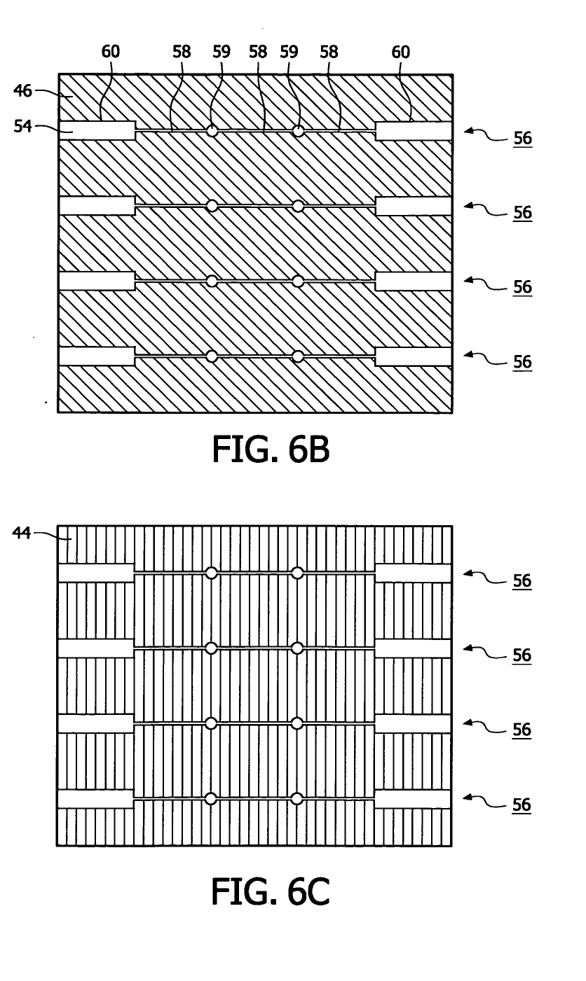

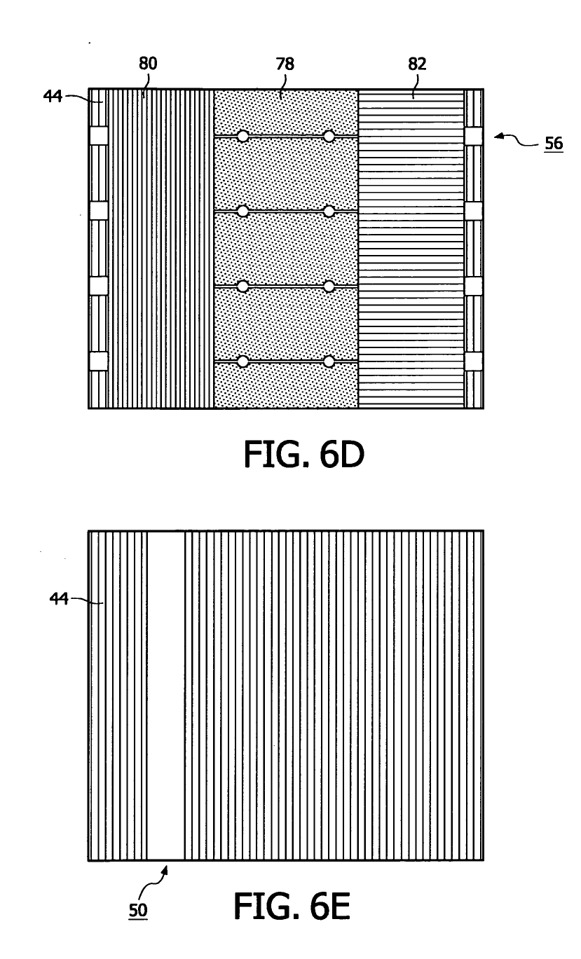

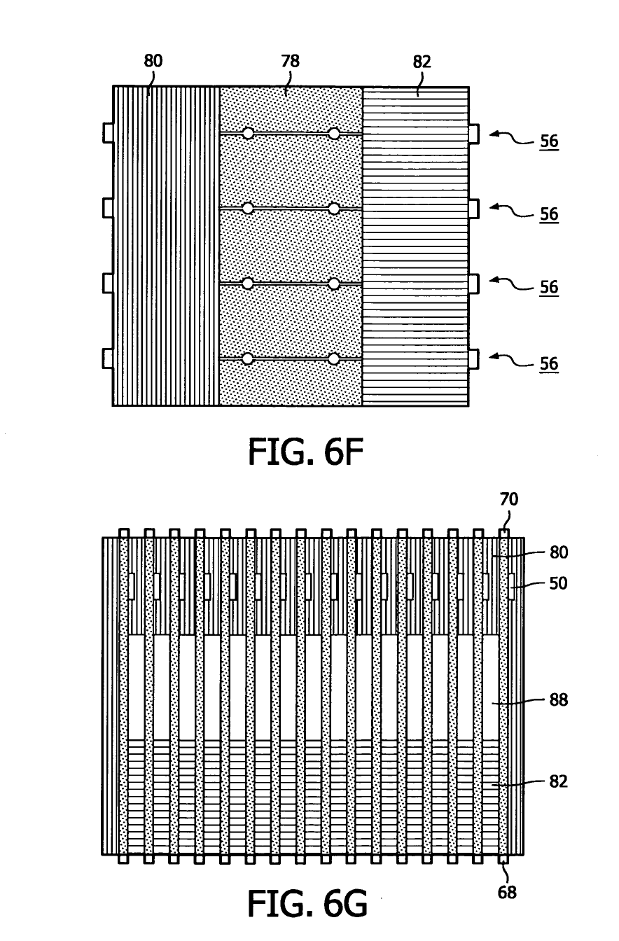

Another exemplary embodiment of an interposer according to the present disclosure is described with reference to FIGS. 2 and 6A-6E. Referring now to FIGS. 2 and 6A, a first nickel/gold layer 48 (e.g., about two microns and one micron in thickness, respectively) is electroplated onto the exposed surface of the first copper layer 46. The first nickel/gold layer 48 forms/defines a heat transporter bar 50. Referring now to FIG. 6B, a second nickel/gold layer 54 (e.g., about 2 microns and one micron in thickness, respectively) is electroplated onto the exposed surface of the second copper layer 42. The second nickel/gold layer 54 forms/defines a plurality of signal tracks 56, each of which includes narrow first portions 58 and wider second and third portions 59, 60.

A first etching process is performed to remove exposed portions of the first copper layer 42, thereby exposing one side of the nickel layer 44, as shown in FIG. 6C. A glue layer 78 is applied to the signal line 56 side of the nickel layer 44. A strip of backing material 80 and a strip of epoxy molding compound 82 are applied to portions of the glue layer 78, as shown in FIG. 6D.

A second etching process may be performed to remove exposed portions of the second copper layer 46, thereby exposing the opposite side of the nickel layer 44, as shown in FIG. 6E. A third etching process is performed to remove the nickel layer 44. A low viscosity epoxy is placed between the heat transporter bar 50 and the first portions 58 of the signal lines 65 to prevent electrical contact between the signal lines 56 and the heat removal bar 50. A layer 84 of the interposer 86 is shown in FIG. 6F. An epoxy that is generally characterized by a low coefficient of thermal expansion is typically used to adhere a plurality of interposer layers 84 to form exemplary interposer 86, as shown in FIG. 6G. It is noted that air gaps 88 are advantageously defined between the layers 84 to create/provide a further thermal barrier.

Contact portions 68, 70 are then added to the interposer assembly 86. The thicknesses of the backing material 80 and epoxy molding compound 82 are selected to space the contact portions 68 in correspondence with contact portions of the flexible interconnection assembly 28 (shown in FIG. 8A) as will be described below in detail. In addition, heat bypasses 34 (shown in FIG. 1) are attached to or otherwise placed in thermal communication with portions of the heat transporter bars 50 that extend past the backing material 80 and the heat sink assembly 32 (shown in FIG. 1).

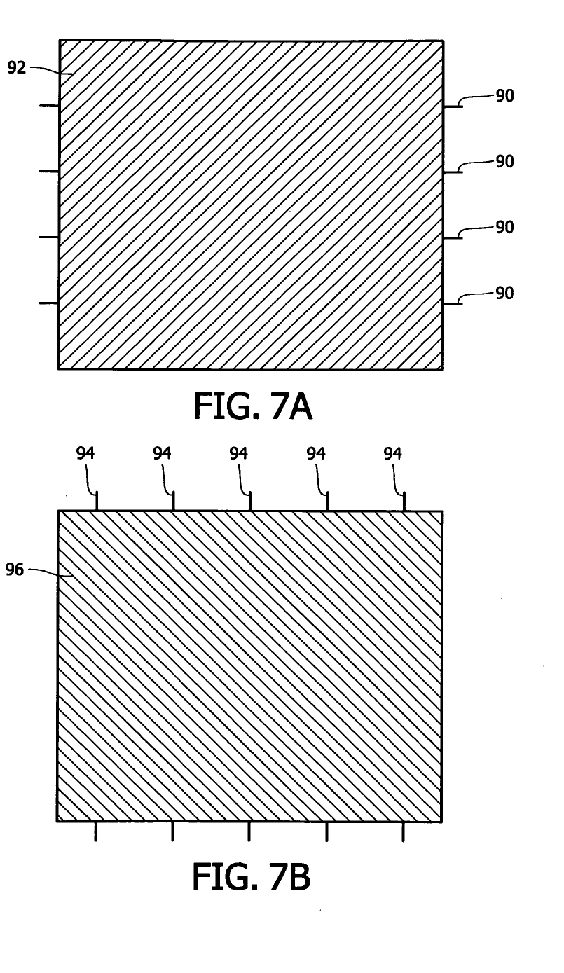

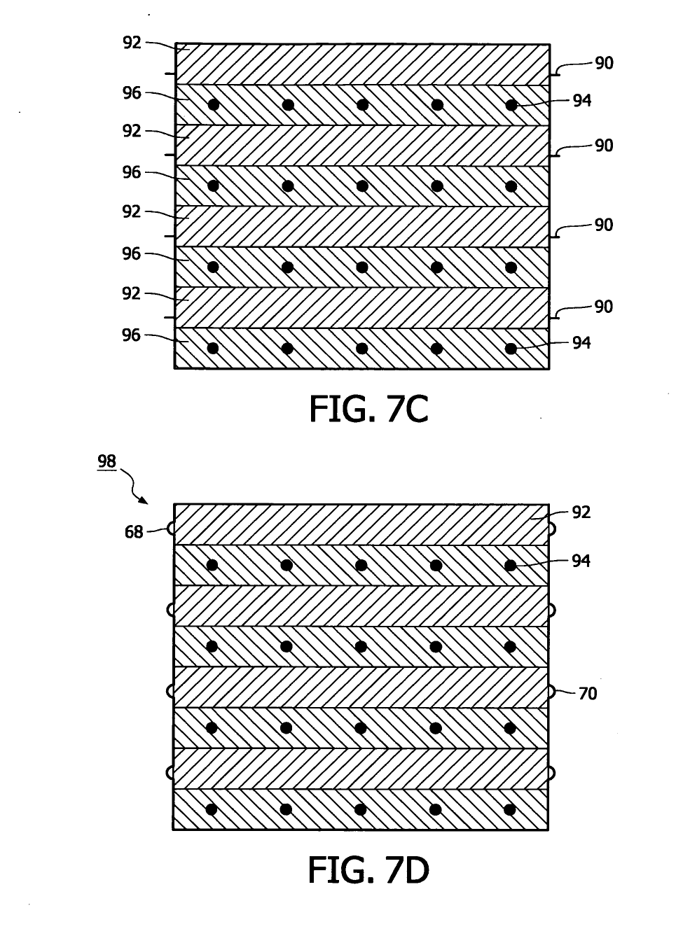

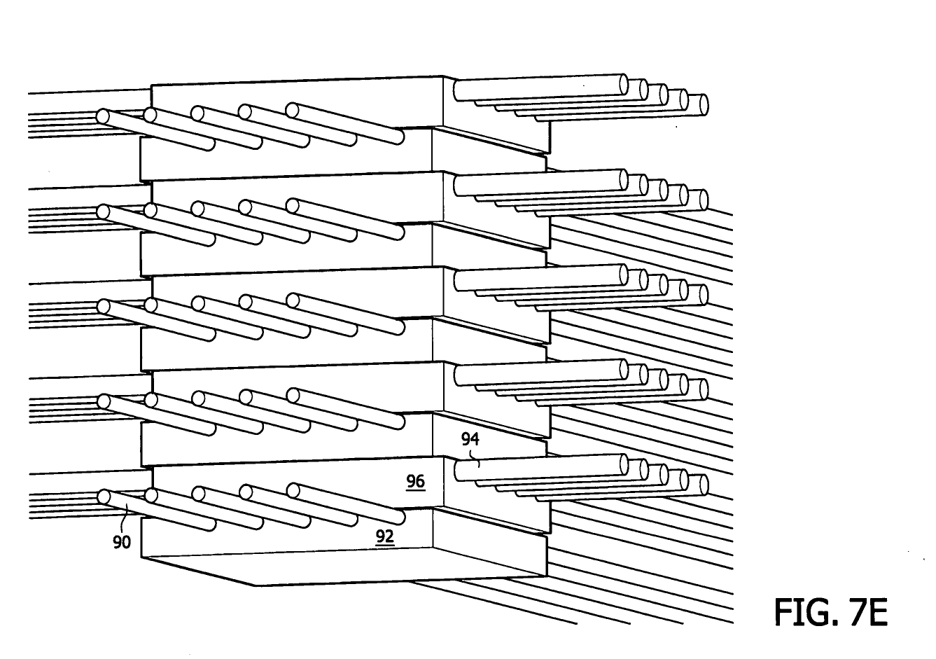

Another embodiment of an exemplary interposer assembly according to the present disclosure is described with reference to FIGS. 7A-7F.

Referring now to FIG. 7A, a plurality of wires 90 are positioned within a layer of backing material 92. Referring now to FIG. 7B, a plurality of wires 94 are formed within a layer of underfill material 96. An epoxy glue is used to adhere a plurality of layers of backing material 92 and layers of underfill material 96 to form an interposer assembly 98, as shown in FIGS. 7C and 7E.

Contact portions 68, 70 are then added to the interposer assembly 98. The thicknesses of the backing material 92 and the underfill material 96 are selected to space the contact portions 68 in correspondence with contact portions of the flexible interconnection assembly 28 (shown in FIG. 8A), as will be described below in detail. In addition, heat bypasses 34 (shown FIG. 1) are attached to portions of the wires 94 that extend past the underfill material 96.

Referring once again to FIGS. 1 and 8A, the interposer assembly 22 of the exemplary ultrasonic transducer probe 10 is interconnected to the transducer control assembly 30 using flexible interconnection assembly 28. The flexible interconnection assembly 28 includes interconnection members 100 having contact portions 102, 104 disposed with respect to opposing surfaces 106, 108 of a flexible member 110. Contact portions 68 of the interposer assembly 22 are aligned with contact portions 102 of the flexible interconnect assembly 28 and contact portions 112 of the transducer control assembly 30 are aligned with contact portions 104 of the flexible interconnect assembly 28, as shown in FIG. 8A. A non-conducting glue is used attach the acoustic stack (not shown) to the interposer assembly 22.

A force F1 is applied to the interposer assembly 22 and a force F2 is applied to transducer control assembly 30. The contact portions 68 of the interposer assembly 22 and the contract portions 112 of the transducer control assembly 30 are not aligned in vertical planes; thus, the application of forces F1, F2 causes the interconnection members 100 to rotate with respect to surfaces 106, 108 of the flexible member 110. Such rotation ensures a good electrical interconnection and compensates for manufacturing variations in heights of the contact portions 68 of the interposer assembly 22 and contact portions 112 of the transducer control assembly 30.

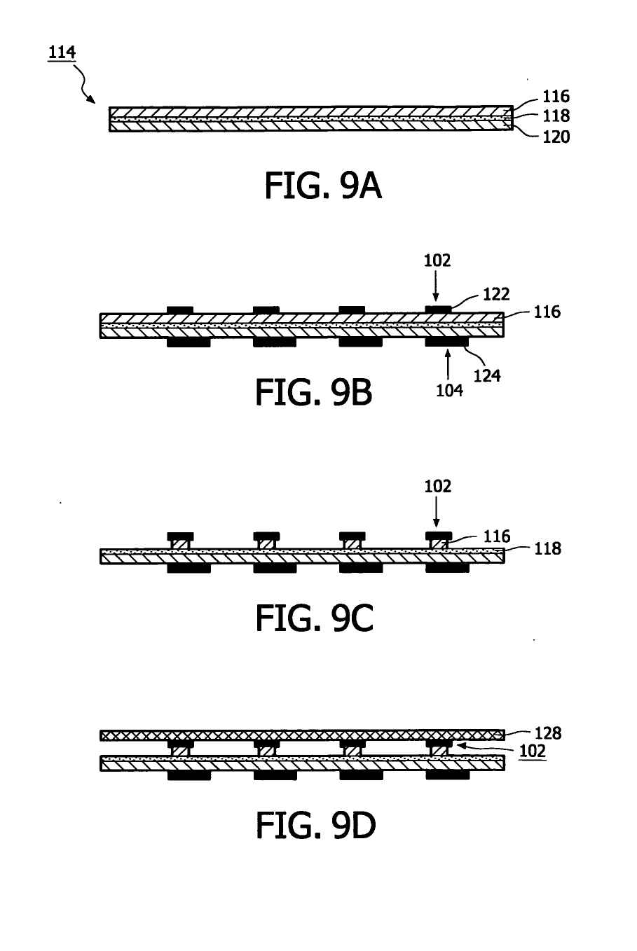

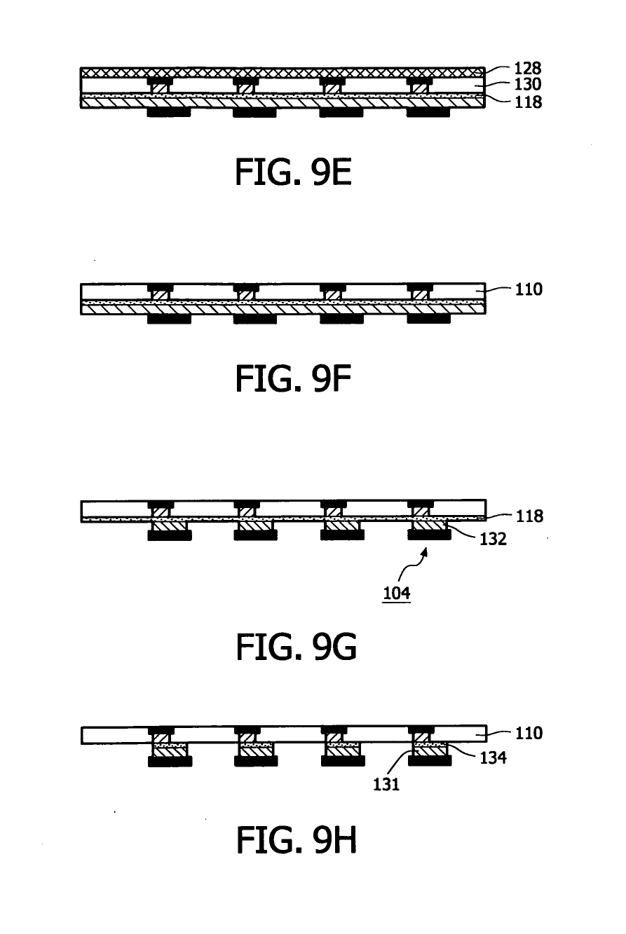

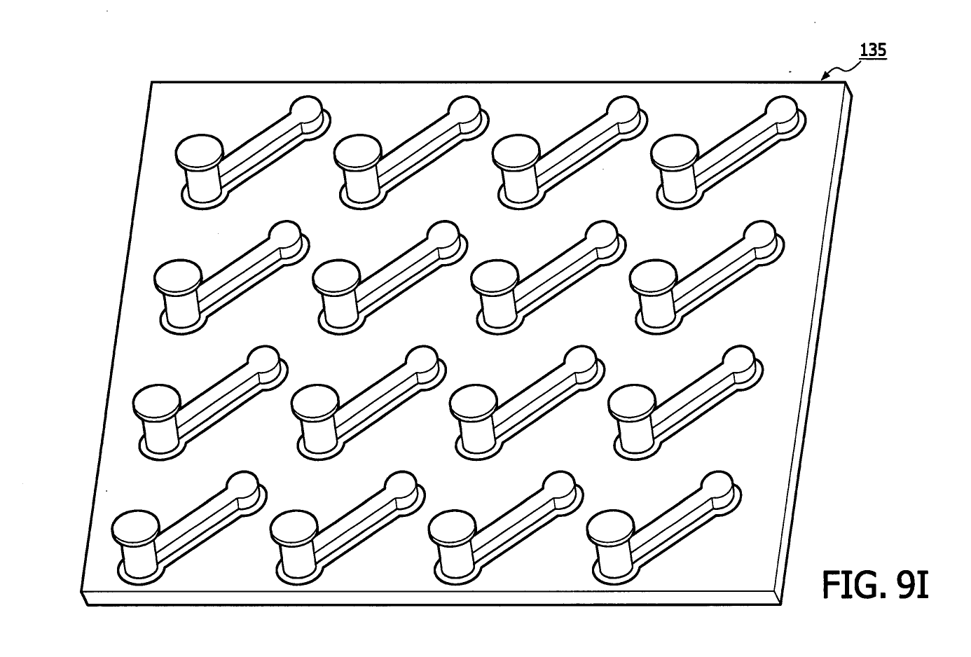

Fabrication of an exemplary flexible interconnect assembly 28 is described with reference to FIGS. 9A-9I. With particular reference to FIG. 9A, fabrication of the interposer assembly 22 may advantageously begin with a metallic stack 114. The metallic stack 114 includes a first copper layer 116 (e.g., about twenty-five microns in thickness), a nickel layer 118 (e.g., about one micron in thickness), and a second copper layer 120 (e.g., about sixty-five microns in thickness).

A first nickel/palladium/gold layer 122 (e.g., about two microns, one micron, and one-half micron in thickness, respectively) is electroplated onto the exposed surface of the first copper layer 116, as shown in FIG. 9B. The first nickel/palladium/gold layer 122 forms contact portions 102. A second nickel/palladium/gold layer 124 (e.g., about two microns, one micron, and one-half micron in thickness, respectively) is electroplated onto the exposed surface of the second copper layer 120, as shown in FIG. 9B. The second nickel/palladium/gold layer 124 forms the contact portions 104.

A first etching process is performed to remove exposed portions of the first copper layer 116 leaving portions 126 of the first copper layer 116 between the contact portions 102 and the nickel layer 118, as shown in FIG. 9C. A tape 128 is placed on the contact portions 102, as shown in FIG. 9D. An elastomeric material 130, such as polydimethylsiloxane (PMDS) elastomer, for example, is provided between the tape 128 and nickel layer 118, as shown in FIG. 9E. The elastomeric material 130 is cured and the tape 128 is removed leaving the flexible member 110, as shown in FIG. 9F.

A second etching process is performed to remove exposed portions of the second copper layer 120 leaving portions 132 of the second copper layer 120 between the contact portions 104 and the nickel layer 118, as shown in FIG. 9G. A third etching process is performed to remove exposed portions of the nickel layer 118 leaving portions 131, as shown in FIG. 9H. A perspective view of an exemplary assembly 135 fabricated according to the foregoing process is depicted in FIG. 9I.

Referring now to FIG. 10A, the transducer control assembly 30 includes contact portions 112 that are connected to individual transducer elements (not shown). The transducer control assembly 30 also includes contact portions 134 that are connected to a processing assembly (not shown) that provides control signals to the transducer control assembly 30. Connection of the transducer control assembly 30 to the processing assembly is described with reference to FIGS. 10B-10C.

A flexible interconnection assembly 140 includes interconnection members 142 having contact portions 144, 146 disposed with respect to opposing surfaces 148, 150 of a flexible member 152. Contact portions 154 of the interposer assembly 22 are aligned with contact portions 144 of the flexible interconnect assembly 140 and contact portions 134 of the transducer control assembly 30 are aligned with contact portions 146 of the flexible interconnect assembly 140, as shown in FIG. 10B. A force F1 is applied to the interposer assembly 22 and a force F2 is applied to the transducer control assembly 30. The contact members 154 of the interposer assembly 22 and the contact portions 134 of the transducer control assembly 30 are not aligned in vertical planes; thus, the application of forces F1, F2 causes the interconnection members 142 to rotate with respect to surfaces 148, 150 of the flexible member 152. Such rotation ensures a good electrical interconnection and compensates for manufacturing variations in heights of the contact portions 134 of the transducer control assembly 30.

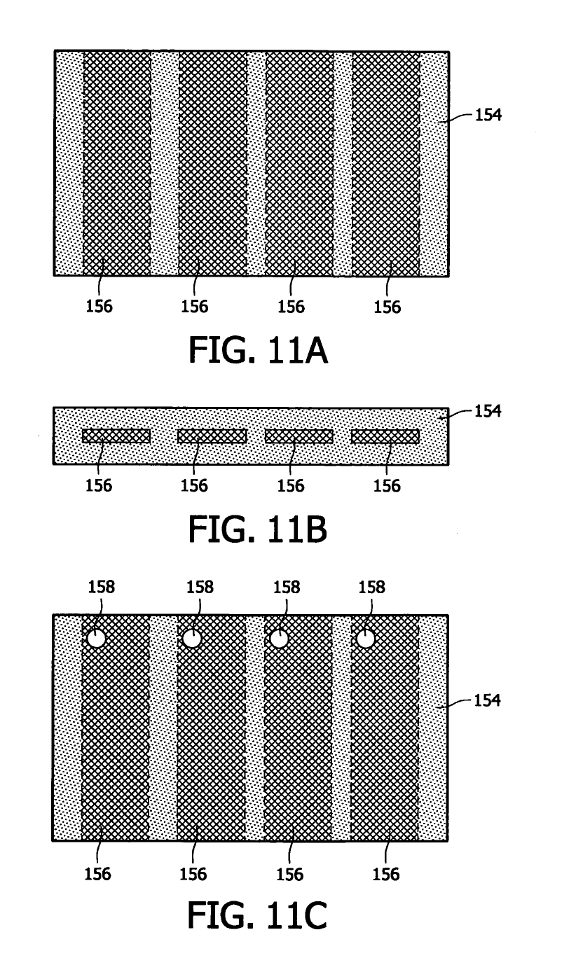

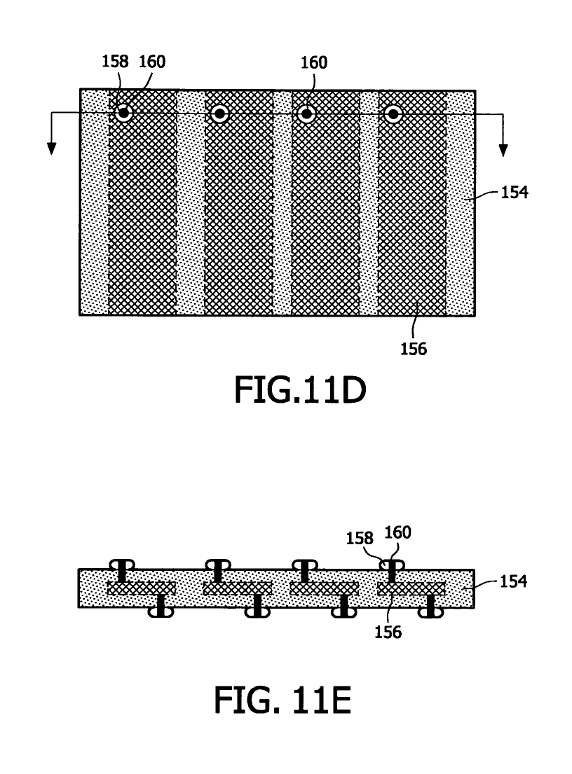

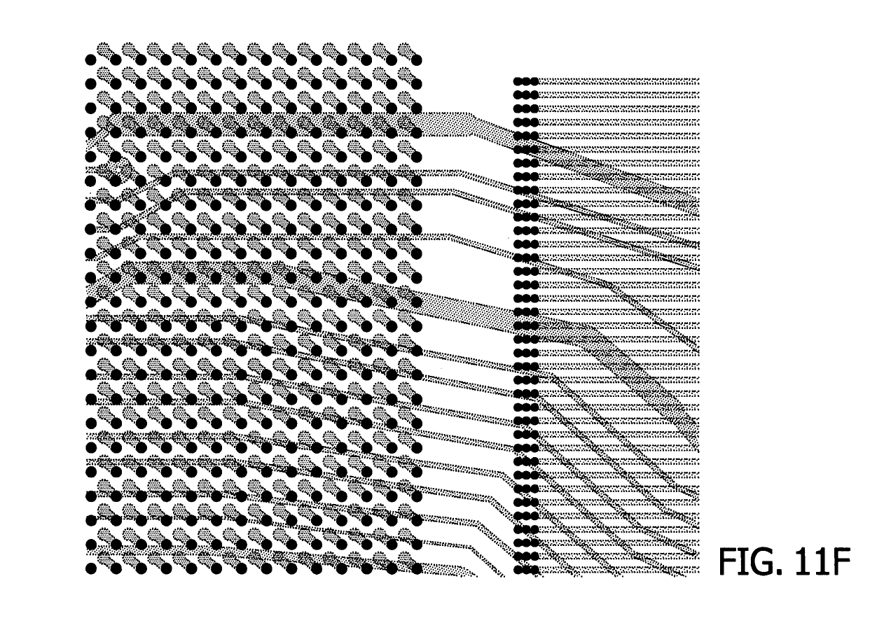

Fabrication of the flexible interconnection assembly 140 is described with reference to FIGS. 11A-11F.

As shown in FIGS. 11A and 11B, a polyimide foil 154 includes copper signal tracks 156 disposed therewithin. Tracks 156 are schematically depicted in FIGS. 11A and 11B. However, as shown in the X-ray image of FIG. 11F, tracks 156 generally take the form of an array of pads. Copper pads 158 are formed on a first surface of the polyimide foil 154 as shown in FIG. 11C. A laser drill (not shown) is used to form vias in the copper pads 158 and portions of the polyimide foil 154 disposed above the signal tracks 156. The vias are filled with copper forming contact members 160 as shown in FIG. 11D. The contact members 160 establish electrical connections between the copper pads 158 and the signals tracks 156. A similar process is repeated on the other side of the polyimide foil 154. A cross section along line 11-11 of a completed interconnection assembly 140 is shown in FIG. 11E. The signal tracks 156 are in communication with connector(s) (not shown) attached to ends opposite the contact members 160. The connector(s) is/are attached to the processing assembly (not shown).

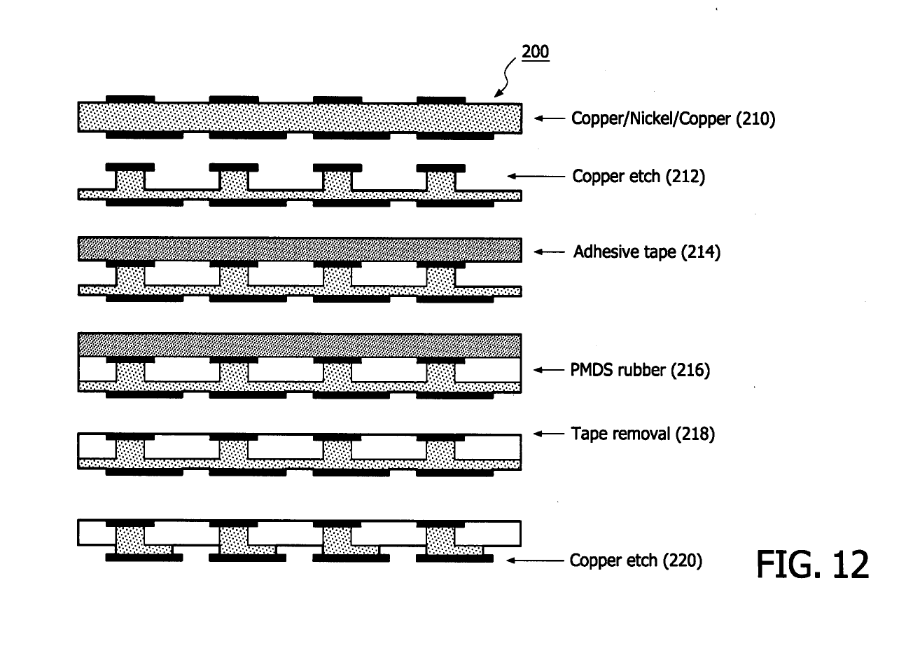

With reference to FIG. 12, a schematic flowchart 200 is depicted. In step 210, a copper/nickel/copper stack is provided with spaced contacts on opposed surfaces thereof. In step 212, a first copper etch is applied to remove a portion of the upper copper layer. In step 214, an adhesive tape is applied across the top of the stack. In step 216, a PMDS rubber is introduced to the void created by the first copper etch and defined below the PMDS rubber. In step 218, the PMDS rubber is removed. In step 220, a second copper etch is undertaken to remove a portion of the lower copper layer, thereby defining electrical communications from contact-to-contact with an intermediate PMDS rubber layer that provides advantageous flexibility to the assembly.

The disclosed assembly defines a “flex-pad” that is advantageously adapted to provide electrical communication between, inter alia, one or more ASICs and an interposer as part of an ultrasound transducer assembly. In an exemplary embodiment, the top contacts and the bottom contact pads are gold-plated.

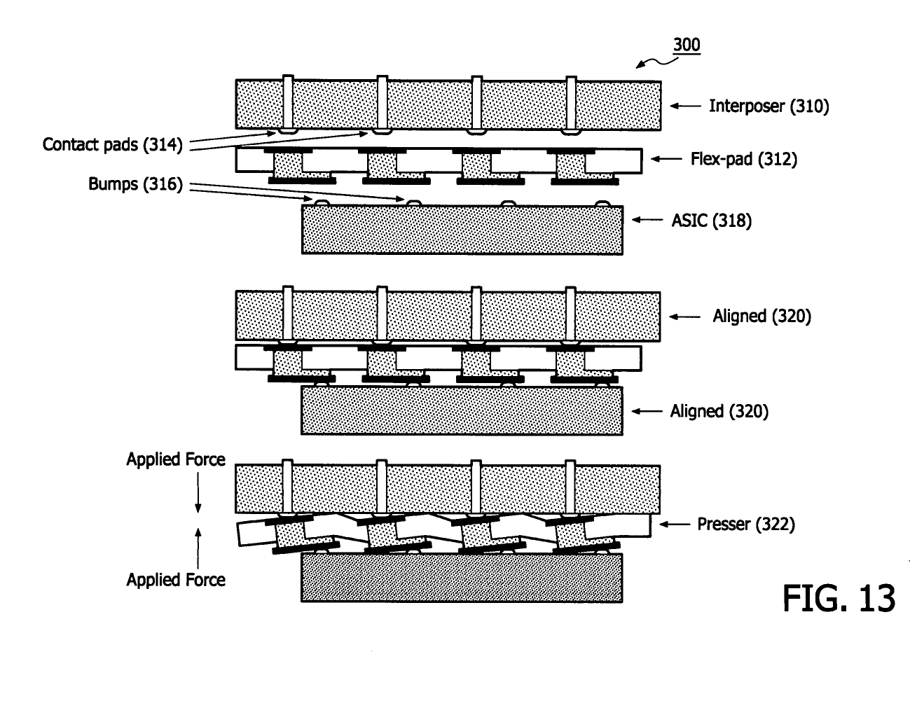

With reference to FIG. 13, a three-step schematic flowchart 300 is provided wherein the assembly of a flex-pad fabricated according to flowchart 200 of FIG. 12 is combined with an ASIC and an interposer to provide reliable, advantageous electrical communications therebetween. As shown in the top portion of FIG. 13, flex-pad 312 is positioned between interposer 310 and ASIC 318. Contact pads 314 defined on interposer 310 are aligned with corresponding top contacts of flex-pad 312, and bumps 316 of ASIC 318 are aligned with corresponding contact strips of flex-pad 312. Thus, as shown in the middle view of FIG. 13, the interposer and the ASIC 318 are brought into contact with flex-pad 312 so as to define an aligned orientation 320. Then, as shown in the bottom view of FIG. 13, a further applied force is delivered to the assembly, thereby flexing flex-pad 312 into a pressed orientation 322. In such pressed orientation 322, reliable electrical communication between ASIC 318 and interposer 310 is established.

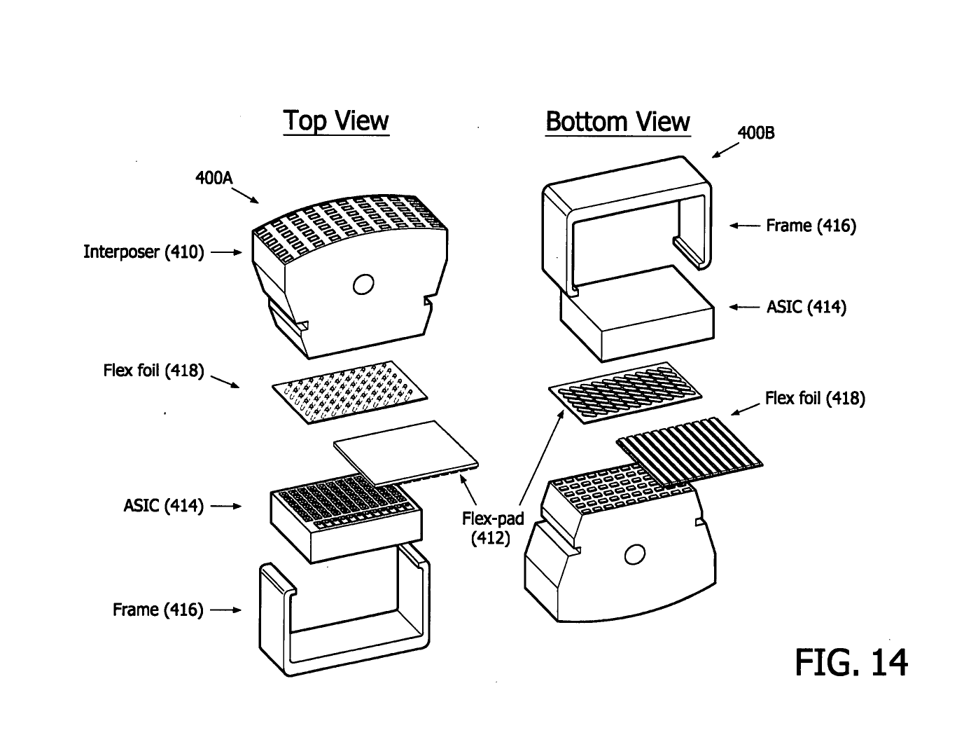

Turning to FIG. 14, top exploded view 400A and bottom exploded view 400B of an exemplary transducer subassembly according to the present disclosure are provided. As shown in FIG. 14, an exemplary interposer includes a substantially curved upper surface with exposed contacts for electrical contact with piezoelectric contacts. Flex-pad 412 is positioned between interposer 410 and ASIC 414. In addition, in the exemplary sub-assembly depicted in FIG. 14, an optional flex foil 418 (e.g., a polyimide film with metalised vias formed with respect thereto to facilitate electrical communication thereacross) is positioned between flex-pad 412 and ASIC 414 to provide greater flexibility and to further facilitate dematability of the disclosed subassembly. A frame 416 with inwardly directed latch arms is adapted to engage slots formed in the side walls of interposer 410 to secure the disclosed components and to supply sufficient compression force to achieve the desired deflection within flex-pad 412. Thus, a conveniently fabricated subassembly for providing reliable electrical communication is provided, such subassembly being easily disassembled, e.g., to provide a substitute ASIC.

Thus, the present disclosure provides advantageous transducer designs and fabrication methods wherein a reliable electrical connection is achieved between an ASIC and an interposer assembly by positioning a flexible film with an array of metal pads therebetween. The flexible film is effective to provide and maintain desired electrical connections because, inter alia, each metal pad in the flexible film is forced to rotate “out of plane” and, as a result, applies a continuous contact force. Individual metal pads may rotate independent from neighboring strips, thereby advantageously compensating for distance variations between contact features/bumps associated with the ASICs and contact features/pads associated with the interposer assembly. While the disclosed “flex-pads” are particularly advantageous in ultrasound transducer applications, the disclosed flex-pads have application in any assembly/design where a pressure contact is desired between spaced arrays of contacts.

Although the present disclosure has been described with reference to exemplary embodiments and exemplary applications, the present disclosure is not limited thereby. Rather, the disclosed apparatus, systems and methods are subject to various changes, modifications, enhancements and/or alternative applications without departing from the spirit or scope of the present disclosure. Indeed, the present disclosure expressly encompasses all such changes, modifications, enhancements and alternative applications herein.

1. A transducer assembly, comprising:
a housing;
an array of transducer elements disposed with respect to said housing;
an interposer assembly disposed with respect to said housing, said interposer assembly including a plurality of signal tracks, a backing material for absorbing acoustic energy generated by the array of transducer elements, and means for transporting heat disposed within said backing material and with respect to said plurality of signal tracks, wherein the heat transporting means is configured to transport heat originating from the plurality of transducer elements away from the transducer elements;
a transducer array control assembly disposed with respect to said housing;
a flex-pad interconnection assembly of electrical contacts configured to provide continuous contact force electrical connections between the plurality of signal tracks of the interposer assembly and electrical contacts of the transducer array control assembly in response to an applied continuous contact force, wherein electrical connections of the flex-pad interconnection assembly are dematable in the absence of the applied continuous contact force;
a heat sink assembly disposed with respect to said transducer array control assembly, wherein the heat sink assembly is configured to conduct heat from the transducer array control assembly; and
a thermal bypass frame means for (i) conducting heat from the heat transporting means of the interposer assembly to the heat sink assembly and (ii) supplying compression force to provide the applied continuous contact force within the flex-pad interconnection assembly disposed between the interposer assembly and the transducer array control assembly.

2. The transducer assembly according to claim 1, wherein the interposer assembly further includes a thermal barrier configured to (i) direct heat from the array of transducer elements to the heat transport means and (ii) to prevent heat generated by the transducer array control assembly from migrating towards the array of transducer elements.

3. The transducer assembly according to claim 1, wherein the means for transporting heat is effective to remove heat generated by acoustic losses in the absence of an ASIC within the housing.

4. The transducer assembly according to claim 1, wherein said signal tracks include first portions and second portions, said first portions having a width that is less than a width of said second portions, and wherein said means for transporting heat is disposed with respect to said first portions.

5. The transducer assembly according to claim 1, further comprising one or more air gaps defined within said interposer assembly for providing a thermal barrier therewithin.

6. The transducer assembly according to claim 5, wherein the plurality of signal tracks extend across the one or more air gaps.

7. The transducer assembly according to claim 6, further wherein the plurality of signal tracks are positioned within a polymeric film.

8. A transducer subassembly, comprising:
an interposer that includes a plurality of contacts defined with respect to an abutment face thereof;
a flex-pad positioned adjacent the interposer, the flex-pad defining a first face and a second face, and including a plurality of electrical contacts associated with each of the first and second faces thereof; and
at least one ASIC adjacent the flex-pad and defining a plurality of contacts with respect to an exposed face thereof;
wherein an applied force is effective to flex the flex-pad so as to establish reliable electrical communication across the flex-pad between the interposer and the at least one ASIC.

9. The transducer subassembly according to claim 8, further comprising a frame that is configured to maintain the applied force on the flex-pad.

10. The transducer subassembly according to claim 8, wherein the flex-pad is fabricated from a copper/nickel/copper substrate.

11. The transducer subassembly according to claim 8, wherein the flex-pad includes a rubber layer between the plurality of contacts defined on the first and second faces thereof.

12. The transducer subassembly according to claim 8, further comprising a flex foil positioned between the interposer and at least one ASIC.

13. The transducer subassembly according to claim 8, wherein the flex-pad facilitates disassembly for removal and/or replacement of the at least one ASIC.

14. A method for fabricating a flex-pad for facilitating electrical communication between spaced contacts, comprising:
providing a metal stack that includes at least two electrically conductive layers;
defining spaced contacts in a predetermined pattern on a first and second face of the metal stack;
performing a first etch process to remove material adjacent the first face, the first etch process leaving substantially undisturbed the metal below the predetermined pattern of contacts defined with respect to the first face;
providing a boundary material with respect to the etched first face;
introducing a rubber material to the region defined by the boundary material and an underlying metal layer;
removing the boundary material; and
performing a second etch process to remove material adjacent the second face, the second etch process leaving substantially undisturbed the metal above the predetermined pattern of contacts defined with respect to the second face, thereby defining a flex-pad.

15. The method of claim 14, wherein the metal stack is a copper/nickel/copper stack.

16. The method of claim 14, wherein the predetermined contacts associated with the second face define contact pads.

17. The method of claim 14, further comprising:
positioning the flex-pad between a first member and a second member, and
applying a compressive force to the flex-pad so as to flex the rubber material associated therewith.

18. The method of claim 17, wherein the first member is an interposer and the second member is at least one ASIC.

19. The method of claim 18, further comprising:
providing a frame to maintain the interposer, the flex-pad and the at least one ASIC in a desired relative position.

20. The method of claim 19, further comprising:
disassembling the at least one ASIC from the assembly by removing the frame and disassociating the at least one ASIC from the flex-pad.

21. The method of claim 17, further comprising positioning a flexible foil member between the flex-pad and the at least one ASIC.
